(19) United States
(12) Patent Application Publication
(10) Pub. No.: US 2024/0139290 A1
(43) Pub. Date: May 2, 2024

(54) METHODS OF TREATING A CARDIOVASCULAR ISCHEMIC EVENT

(71) Applicant: Radius Health, Inc., Boston, MA (US)

(72) Inventors: Beate Klara Maria Mannstadt, Arlington, MA (US); Bruce Mitlak, Boston, MA (US)

(73) Assignee: Radius Health, Inc., Boston, MA (US)

(21) Appl. No.: 18/405,807

(22) Filed: Jan. 5, 2024

Related U.S. Application Data
(63) Continuation of application No. PCT/IB2022/056306, filed on Jul. 7, 2022.
(60) Provisional application No. 63/219,212, filed on Jul. 7, 2021.

(51) Int. Cl. A61K 38/29; A61P 9/10

(52) U.S. Cl. CPC A61K 38/29; A61P 9/10

(57) ABSTRACT

Disclosed herein is a method of treating cardiomyocyte reperfusion injury associated with an ischemic myocardial event. The method includes administering to a subject in need thereof an effective amount of a abaloparatide to a subject in need thereof prior to performing reperfusion, concurrent with performing reperfusion, or both. Further provided is a method of treating cardiomyocyte reperfusion injury associated with cardiac transplant. The method includes contacting a donor heart with an effective amount of abaloparatide.

### CROSS-REFERENCE TO RELATED APPLICATIONS

This application is a continuation of International Application No. PCT/IB2022/056306, filed Jul. 7, 2022, and claims the benefit of U.S. Provisional Application No. 63/219,212, filed on Jul. 7, 2021, the contents of each of which are herein incorporated by reference in their entireties.

### SEQUENCE LISTING

The instant application contains a Sequence Listing which has been submitted electronically in XML format and is hereby incorporated by reference in its entirety. Said CRF copy, created on Sep. 21, 2022, is named SL1190.xml and is 2,687 bytes in size.

### TECHNICAL FIELD

The present disclosure generally relates to methods of treating cardiomyocyte reperfusion injury associated with an ischemic myocardial event.

### BACKGROUND

Cardiac ischemia, a condition characterized by reduced blood flow and oxygen to the myocardium (heart muscle), is one hallmark of cardiovascular disease that can ultimately lead to an acute myocardial infarction (AMI; “heart attack”). AMI remains one of the leading causes of death and disability worldwide. The current standard of care for AMI is timely reperfusion (re-establishment of blood flow and re-oxygenation of the affected area), thereby limiting the temporal progression of ischemia-induced cardiomyocyte death and reducing myocardial infarct size. Despite the benefits of timely restoration of blood flow to ischemic myocardium, paradoxically, such restoration is associated with an element of cardiomyocyte death that may be responsible for as much as 50% of the final infarct size—a phenomenon termed lethal reperfusion injury. Despite optimal myocardial reperfusion, the rate of death after an acute myocardial infarction may reach 10%, and the incidence of cardiac failure after an acute myocardial infarction may reach 25% (Yellon et al., “Myocardial Reperfusion Injury” New England Journal of Medicine 357; 11, 2007).

Mechanisms for reperfusion injury are complex and include oxidative stress, intracellular calcium overload, rapid restoration of physiologic pH, and inflammation which each interact to mediate cardiomyocyte death through mitochondrial injury via opening of the mitochondrial permeability transition pore (mPTP) (Yellon et al., 2007). Accordingly, efforts have been made to mitigate reperfusion injury via administration of pharmacologic agents including reactive oxygen species scavengers, calcium channel antagonists, and inhibitors of mPTP opening. While generally promising in preclinical studies, such initial promise has not been born out in subsequent clinical trials. Indeed, none of these strategies have been successfully translated from preclinical models into clinical practice (Yellon et al., 2007).

Accordingly, it would be desirable to provide a method of treating reperfusion injury associated with ischemic cardiac events.

### SUMMARY OF THE DISCLOSURE

It is known in the art that elevated and persistent levels of parathyroid hormone (PTH) signaling, as seen with hyperparathyroidism and/or hypercalcemia of malignancy, are associated with adverse effects on cardiovascular health. For example, a high level of PTH, such as that seen in primary and secondary hyperparathyroidism, is associated with an increased prevalence of hypertension, left ventricular hypertrophy, heart failure, cardiac arrhythmias, and valvular calcific disease, which may contribute to higher cardiac morbidity and mortality. (Brown et al., “The Parathyroid Gland and Heart Disease,” Methodist Debakey Cardiovasc J. 2017 April-June; 13(2): 49-54). Parathyroid hormone-related protein (PTHrP; UniProt Accession No. P12272) shares some homology with parathyroid hormone (PTH) at the N-terminal end, and both proteins bind to the same G-protein coupled receptor, PTH receptor type-1 (PTH1R). In recent years, PTHrP and its secretory forms (PTHrP(1-36), PTHrP(38-94), and osteostatin), as well as analogues thereof, have been investigated as potential treatments for osteoporosis.

Teriparatide is a truncated recombinant version of human PTH (hPTH(1-34)). A product containing teriparatide, formulated for subcutaneous injection and used for treatment of osteoporosis in postmenopausal women, is available under the brand name FORTEO™, marketed by Eli Lilly, Inc. Clinical studies have demonstrated transient episodes of symptomatic orthostatic hypotension as well as palpitations and tachycardia associated with teriparatide.

Abaloparatide is a synthetic 34 amino acid peptide analog of human PTHrP(1-34) having 76% homology to human PTHrP(1-34) and 41% homology to human PTH(1-34). A product containing abaloparatide, formulated for subcutaneous injection and used for treatment of osteoporosis in postmenopausal women, is available under the brand name TYMLOS®, marketed by Radius Health Inc. In clinical studies conducted on abaloparatide in support of regulatory approval for treatment of postmenopausal women with osteoporosis in Europe, subcutaneous injection of abaloparatide was associated with transient increases in heart rate and higher incidences of palpitations, relative to placebo. At least partially on the basis of purported cardiovascular risk, the European Medicines Agency and the Committee for Medicinal Products for Human Use (CHMP) issued a negative opinion for granting marketing authorization to abaloparatide.

Surprisingly, despite the prior cardiovascular observations potentially associated with abaloparatide, it has been discovered according to the present disclosure that abaloparatide actually possesses a cardioprotective effect. Specifically, as disclosed herein, both abaloparatide and PTHrP(1-36) demonstrated an enhancement in viability of HL-1 cardiomyocyte cells (an immortal murine atrial cell line) in an in vitro model of lethal ischemia-reperfusion injury. On the basis of the in vitro model results, the efficacy of abaloparatide appears to be slightly but significantly greater than that of PTHrP(1-36). Such results support the cardioprotective potential of abaloparatide in ischemia-reperfusion scenarios.

Accordingly, in one aspect is provided a method of treating cardiomyocyte reperfusion injury associated with an ischemic myocardial event, the method comprising administering to a subject in need thereof an effective amount of abaloparatide prior to performing reperfusion, concurrent with performing reperfusion, or prior to performing reperfusion and concurrently with performing reperfusion.

In some embodiments, the ischemic myocardial event is an acute myocardial infarction, atherosclerosis, coronary artery spasm, cardiopulmonary bypass, thromboembolism, or cardiac transplant surgery, and wherein the ischemic myocardial event comprises at least a partial occlusion of one or more coronary arteries.

In some embodiments, the reperfusion comprises angioplasty, coronary artery bypass surgery, or reversal of cardiopulmonary bypass.

In some embodiments, the administration is subcutaneous, intravenous, or intracoronary. In some embodiments, the administration is transdermal (e.g., using a patch). In some embodiments, the abaloparatide is administered as an intramuscular, intradermal, or subcutaneous depot formulation.

In some embodiments, the abaloparatide is administered from about 1 minute to about 1 hour, or from about 1 hour to about 12 hours, or from about 12 hours to about 24 hours, or from about 24 hours to about 7 days prior to performing the reperfusion. In some embodiments, the abaloparatide is administered once daily for about 1 day to about 7 days prior to performing reperfusion.

In some embodiments, the abaloparatide is administered concurrently with performing reperfusion.

In some embodiments, the abaloparatide is dosed subcutaneously in an amount from about 20 μg to about 400 μg. In some embodiments, the abaloparatide is dosed subcutaneously in an amount of about 80 μg.

In some embodiments, the abaloparatide is administered using a transdermal microneedle patch in an amount from about 100 μg to about 300 μg.

In some embodiments, the abaloparatide is administered intravenously at a dose sufficient to provide a maximum exposure of about 240 pmol/kg·min.

In some embodiments, the subject is identified as at risk for myocardial infarction.

In some embodiments, an expression level of phosphorylated Akt, phosphorylated ERK, or both, is enhanced relative to a corresponding expression level in the absence of the administration of abaloparatide.

In another aspect is provided a method of treating cardiomyocyte reperfusion injury associated with cardiac transplant, the method comprising contacting a donor heart with an effective amount of abaloparatide prior to performing reperfusion on said donor heart, concurrent with performing reperfusion on said donor heart, or prior to performing reperfusion on said donor heart and concurrently with performing reperfusion on said donor heart. In some embodiments, contacting comprises perfusing the donor heart with a composition comprising abaloparatide prior to revitalization.

### BRIEF DESCRIPTION OF THE FIGURES

FIG. 1A is a chart showing the percent viability of HL-1 cardiomyocytes under normoxic conditions in the presence of vehicle (control) or abaloparatide at various concentrations.

FIG. 1B is a chart showing the percent viability of HL-1 cardiomyocytes following simulated ischemia-reperfusion in the presence of vehicle or abaloparatide at various concentrations.

FIG. 2A is a chart showing the percent viability of HL-1 cardiomyocytes under normoxic conditions in the presence of vehicle or human parathyroid hormone-related protein PTHrP(1-36) at various concentrations.

FIG. 2B is a chart showing the percent viability of HL-1 cardiomyocytes following simulated ischemia-reperfusion in the presence of vehicle or human parathyroid hormone-related protein PTHrP(1-36) at various concentrations.

FIG. 3A is a photographic image of immunoblotting results depicting the extent of phosphorylation of Akt and ERK in HL-1 cardiomyocytes under normoxic and hypoxia-reoxygenation conditions when exposed to vehicle, abaloparatide, or PTHrP(1-36).

FIG. 3B is a chart illustrating the quantified phosphorylation of Akt in HL-1 cardiomyocytes under hypoxia-reoxygenation conditions when exposed to vehicle, abaloparatide, or PTHrP(1-36).

FIG. 3C is a chart illustrating the quantified phosphorylation of ERK in HL-1 cardiomyocytes under hypoxia-reoxygenation conditions when exposed to vehicle, abaloparatide, or PTHrP(1-36).

FIG. 4A is a chart showing the percent viability of HL-1 cardiomyocytes under normoxic conditions in the presence of vehicle, abaloparatide, or human parathyroid hormone-related protein PTHrP(1-36) at various concentrations.

FIG. 4B is a chart showing the percent viability of HL-1 cardiomyocytes following simulated ischemia-reperfusion in the presence of vehicle, abaloparatide, or human parathyroid hormone-related protein PTHrP(1-36) at various concentrations.

FIG. 5A is a chart showing the percent viability of HL-1 cardiomyocytes under normoxic conditions in the presence of vehicle or abaloparatide at various concentrations, in the presence or absence of PD98059, a pharmacologic inhibitor of MEK-ERK.

FIG. 5B is a chart showing the percent viability of HL-1 cardiomyocytes following simulated ischemia-reperfusion in the presence of vehicle or abaloparatide at various concentrations, in the presence or absence of PD98059.

FIG. 6A is a photographic image of immunoblotting results depicting the extent of phosphorylation of ERK in HL-1 cardiomyocytes under normoxic and hypoxia-reoxygenation conditions when exposed to vehicle, abaloparatide, or PTHrP(1-36), in the presence or absence of PD98059.

FIG. 6B is a chart illustrating the quantified phosphorylation of ERK in HL-1 cardiomyocytes under hypoxia-reoxygenation conditions when exposed to vehicle, abaloparatide, or PTHrP(1-36) in the presence or absence of PD98059.

FIG. 7 is a chart illustrating the concentration of PTHrP(1-36) in plasma over time following a subcutaneous injection in female pigs.

FIG. 8A is a chart illustrating the pulse rate of control and female swine following subcutaneous injection of PTHrP(1-36) at baseline and at various times during reperfusion.

FIG. 8B is a chart illustrating the systolic pressure of control and female swine following subcutaneous injection of PTHrP(1-36) at baseline and at various times during reperfusion.

FIG. 8C is a chart illustrating the diastolic pressure of control and female swine following subcutaneous injection of PTHrP(1-36) at baseline and at various times during reperfusion.

FIG. 9A is a photographic image of a female pig heart from a subject in the control group showing the area at risk of infarct following left anterior descending (LAD) coronary artery occlusion and reperfusion.

FIG. 9B is a photographic image of a female pig heart from a subject in the control group showing the area of necrosis and viable myocardium following LAD coronary artery occlusion and reperfusion.

FIG. 10A is a photographic image of a female pig heart from a subject in the PTHrP(1-36) treated group showing the area at risk of infarct following left anterior descending (LAD) coronary artery occlusion and reperfusion.

FIG. 10B is a photographic image of a female pig heart from a subject in the PTHrP(1-36) treated group showing the area of necrosis and viable myocardium following LAD coronary artery occlusion and reperfusion (best outcome).

FIG. 11A is a photographic image of a female pig heart from another subject in the PTHrP(1-36) treated group showing the area at risk of infarct following left anterior descending (LAD) coronary artery occlusion and reperfusion (worst outcome).

FIG. 11B is a photographic image of a female pig heart from another subject in the PTHrP(1-36) treated group showing the area of necrosis and viable myocardium following LAD coronary artery occlusion and reperfusion.

FIG. 12 is a chart showing mean infarct size in percent of the risk region for animals in the control group and the PTHrP(1-36) treated group.

### DETAILED DESCRIPTION OF DISCLOSURE

The present disclosure generally provides a method for preventing reperfusion injury following an ischemic event. Examples of ischemic events include, but are not limited to, acute myocardial ischemia, myocardial infarction, stroke, and organ transplantation. The method generally comprises administering to a subject in need thereof a therapeutically effective amount of a parathyroid hormone receptor-1 (PTHR1) agonist prior to performing reperfusion, concurrent with performing reperfusion, or both.

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

### Definitions

While the terms used herein are believed to be well understood by one of ordinary skill in the art, definitions are set forth herein to facilitate explanation of the presently-disclosed subject matter.

The articles “a” and “an” are used herein to refer to one or to more than one (i.e., to at least one) of the grammatical object of the article.

The term “about” as used throughout this specification is used to describe and account for small fluctuations. For example, the term “about” can refer to less than or equal to ±5%, such as less than or equal to ±2%, less than or equal to ±1%, less than or equal to ±0.5%, less than or equal to ±0.2%, less than or equal to ±0.1% or less than or equal to ±0.05%. All numeric values herein are modified by the term “about,” whether or not explicitly indicated. A value modified by the term “about” of course includes the specific value. For instance, “about 5.0” must include 5.0.

The term “ischemia” or “ischemic” as used herein refers to an inadequate blood supply to an organ or part of the body, including, but not limited to, the heart. Ischemia may occur through partial or complete blockages of blood flow associated with blood clots, coronary artery disease, injury, or mechanical means such as bypass, balloon procedures, and the like.

The term “reperfusion” as used herein refers to the action of restoring the flow of blood to an organ or tissue, including, but not limited to, the heart. Reperfusion of e.g., coronary flow is necessary to resuscitate the ischemic or hypoxic myocardium, decreasing cardiac morbidity and mortality. Reperfusion may be performed by percutaneous or surgical revascularization procedures, including but not limited to, stents, balloon angioplasty, thrombolysis, bypass surgery, resuscitation, or any other means of restoring blood flow.

The term “revitalization” as used herein refers to reestablishing heart beat and blood flow to the donor heart following a heart transplant procedure.

The term “subject in need thereof” as used herein refers to a mammalian subject, e.g., a human. In some embodiments, the subject in need is a male or female human subject. In some embodiments, the subject in need is at risk of myocardial ischemia, or has myocardial ischemia.

For the purposes of this disclosure, the therapeutic utility of these compounds includes “treating” a human and methods of treatment or treating a subject, human or patient, where treating is understood to include treating, preventing, or ameliorating the symptoms associated with, or reducing the incidence of, reducing the pathogenesis of, facilitating the recovery from or delaying the onset of the syndrome, illness, malady or condition being considered. As it pertains to treating cardiomyocyte reperfusion injury, a method of treatment should be understood to include a method of preventing reperfusion injury.

### Parathyroid Hormone

Parathyroid hormone (PTH) is an 84 amino acid polypeptide hormone secreted by the parathyroid glands. PTH primarily regulates calcium and phosphate concentrations in bone and blood. Parathyroid hormone 1 receptors (PTH1R), present at high levels on the cells of bone and kidney, are activated by the 34 N-terminal amino acids of PTH. Truncated human PTH (hPTH(1-34)), possessing the 34 N-terminal amino acids of PTH is referred to as teriparatide. A recombinant version of teriparatide, formulated for subcutaneous injection, is marketed under the brand name FORTEO™ for treatment of osteoporosis in postmenopausal women.

### Parathyroid Hormone-Related Peptide and Abaloparatide

Parathyroid hormone-related protein (PTHrP) is a protein member of the parathyroid hormone family which acts as an endocrine, autocrine, paracrine, and intracrine hormone. PTHrP exists in several isoforms, ranging in size from 139 to 173 amino acids, as well as multiple secretory forms (PTHrP(1-36), PTHrP(38-94), and osteostatin). See, e.g., Phibrick et al., “Defining the Roles of Parathyroid Hormone-Related Protein in Normal Physiology,” Physiological Reviews 1996, 76(1), 127-173.

The physiological role of PTHrP can be divided into 5 categories: 1) transepithelial calcium transport; 2) smooth muscle relaxation in the uterus, bladder, gastrointestinal tract, and arterial wall; 3) regulation of cellular proliferation; 4) cellular differentiation and apoptosis of multiple tissues; and 5) as an indispensable component of successful pregnancy and fetal development. Parathyroid hormone-related protein (PTHrP) shares some homology with parathyroid hormone (PTH) at the N-terminal end, and both peptides bind to the same G-protein coupled receptor, PTH receptor type-1 (PTH1R). Despite a common receptor, PTH primarily acts as an endocrine regulator of calcium homeostasis, whereas PTHrP plays a fundamental paracrine role in the mediation of endochondral bone development (Kronenberg, “PTHrP and skeletal development,” Ann. NY Acad. Sci. 1068:1-13 (2006)). PTHrP is also expressed in normal fetal and adult heart tissues and in large vessels, and plays a role in cardiovascular development (see, e.g., Towler, D. The Parathyroids, Ch. 12, 3^{rd} edition 2015; https://doi.org/10.1016/B978-0-12-397166-1.00012-6).

The agonistic activation of PTH1R (e.g., with PTHrP, secretory forms, and analogs) results in the production and secretion of osteoclast-stimulating cytokines, while PTH1R antagonists may be used to treat bone metastases, hypercalcemia, cachexia and hyperparathyroidism. In recent years, PTHrP and its secretory forms (PTHrP(1-36), PTHrP(38-94), and osteostatin), as well as analogues thereof, have been investigated as potential treatments for osteoporosis. Subcutaneous injection of PTHrP and its derivatives and analogues has been reported to be effective for treating osteoporosis and/or improving bone healing (Horwitz et al., “Parathyroid hormone-related protein for the treatment of postmenopausal osteoporosis: defining the maximal tolerable dose,” J Clin Endocrinol Metab 95:1279-1287 (2010); Horwitz et al., “Safety and tolerability of subcutaneous PTHrP(1-36) in healthy human volunteers: a dose escalation study,” Osteoporos Int 17:225-230 (2006); Bostrom et al., “Parathyroid hormone-related protein analog RS-66271 is an effective therapy for impaired bone healing in rabbits on corticosteroid therapy,” Bone 26:437-442 (2000); Augustine et al., “Parathyroid hormone and parathyroid hormone-related protein analogs as therapies for osteoporosis,” Curr Osteoporos Rep 11:400-406 (2013)).

The parathyroid hormone-related protein family member having 34 amino acids (PTHrP(1-34)) was originally identified from tumors associated with hypercalcemia. The sequence of native human PTHrP(1-34) is as follows:
Ala¹-Val-Ser-Glu-His⁵-Gln-Leu-Leu-His-Asp¹⁰-Lys-Gly-Lys-Ser-Ile¹⁵-Gln-Asp-Leu-Arg-Arg²⁰-Arg-Phe-Phe-Leu-His²⁵-His-Leu-Ile-Ala-Glu³⁰-Ile-His-Thr-Ala³⁴ (SEQ ID NO:1).

Abaloparatide is a synthetic 34 amino acid peptide analog of human PTHrP(1-34) having 76% homology to human PTHrP(1-34) and 41% homology to human PTH(1-34). The sequence of abaloparatide is as follows:
Ala¹-Val-Ser-Glu-His⁵-Gln-Leu-Leu-His-Asp¹⁰-Lys-Gly-Lys-Ser-Ile¹⁵-Gln-Asp-Leu-Arg-Arg²⁰-Arg-Glu-Leu-Leu-Glu²⁵-Lys-Leu-Leu-Aib-Lys³⁰-Leu-His-Thr-Ala³⁴-NH₂ (SEQ. ID NO: 2).

The structure and preparation of abaloparatide has been previously disclosed in, for example, U.S. Pat. No. 5,969,095, which is hereby incorporated by reference. Abaloparatide has shown potent anabolic activity with decreased bone resorption, less calcium-mobilizing potential, and improved room temperature stability (Obaidi et al., “Pharmacokinetics and Pharmacokinetics and pharmacodynamic of subcutaneously (SC) administered doses of BA058, a bone mass density restoring agent in healthy postmenopausal women,” AAPS Abstract W5385 (2010)). A product containing abaloparatide, formulated for subcutaneous injection and used for treatment of osteoporosis in postmenopausal women, is available under the brand name TYMLOS®, marketed by Radius Health Inc.

### Reperfusion Injury

Myocardial reperfusion injury was first postulated in 1960 by Jennings et al. in their description of the histologic features of reperfused ischemic canine myocardium (Jennings et al., “Myocardial necrosis induced by temporary occlusion of a coronary artery in the dog. Arch Pathol. 1960; 70:68-78). They reported cell swelling, contracture of myofibrils, disruption of the sarcolemma, and the appearance of intramitochondrial calcium phosphate particles. The injury to the heart during myocardial reperfusion causes four types of cardiac dysfunction. Notably, there is significant overlap between these four types of cardiac dysfunction such that they often present in various combinations.

The first is myocardial stunning, referring to the “mechanical dysfunction that persists after reperfusion despite the absence of irreversible damage and despite restoration of normal or near-normal coronary flow.” The myocardium usually recovers from this reversible form of injury after several days or weeks.

The second type of cardiac dysfunction, the “no-reflow phenomenon,” the inability to reperfuse a previously ischemic region, refers to the impedance of microvascular blood flow encountered during opening of the infarct-related coronary artery. Krug A, et al., “Blood supply of the myocardium after temporary coronary occlusion. Circ. Res 1966; 19:57-62; Ito H. “No-reflow phenomenon and prognosis in patients with acute myocardial infarction. Nat Clin Pract Cardiovasc Med 2006; 3:499-506).

The third type of cardiac dysfunction, reperfusion arrhythmias, is potentially harmful, but effective treatments are available. (Manning et al., “Reperfusion-induced arrhythmias: mechanisms and prevention” J Mol Cell Cardiol 1984; 16:497-518).

The last type is reperfusion injury (cell death associated with transient ischemia). It is therefore the component of cell death occurring as a consequence of reperfusion. The most convincing means of demonstrating the existence of reperfusion injury as a distinct mediator of cardiomyocyte death is to show that the size of a myocardial infarct can be reduced by an intervention used at the beginning of myocardial reperfusion. (Yellon et al., 2007)

PTHrP has previously been investigated in models of ischemia-reperfusion or hypoxia-reoxygenation. There is an overall agreement that direct administration of PTHrP, or increased cardiac expression of PTHrP-PTH1R as a secondary consequence of thyroxine-induced hyperthyroidism, confers a protective effect in ischemia-reperfusion. (See, e.g., Jansen et al. “Parathyroid hormone-related peptide improves contractile function of stunned myocardium in rats and pigs,” Am J Physiol Heart Circ Physiol. 2003, 284(1):H49-H55; Grohe et al. “Sex-specific differences in ventricular expression and function of parathyroid hormone-related peptide,” Cardiovasc Res. 2004; 61(2):307-316; Halapas et al. “Experimental hyperthyroidism increases expression of parathyroid hormone-related peptide and type-1 parathyroid hormone receptor in rat ventricular myocardium of the Langendorff ischaemia-reperfusion model,” Exp Physiol. 2008; 93(2): 237-246; Lutteke et al., “Parathyroid hormone-related peptide improves contractile responsiveness of adult rat cardiomyocytes with depressed cell function irrespectively of oxidative inhibition,” Basic Res Cardiol. 2005; 100(4):320-327). However, the aforementioned studies have focused on recovery of contractile function following relief of ischemia or other stressors rather than the potential to reduce infarct size.

As disclosed herein, the PTH1R agonist PTHrP(1-36) reduces infarct size in pigs following left anterior descending (LAD) coronary artery occlusion and reperfusion. Specifically, with reference to Example 7, the infarct sizes ranged from 27% to 66% of the risk region in the control group and from 3% to 26% of the area at risk in pigs that received PTHrP(1-36). Further, as disclosed herein with reference to Examples 1-3, both abaloparatide and PTHrP(1-36) attenuated HL-1 cardiomyocyte death caused by simulated ischemia-reperfusion via up-regulation of classic survival kinase signaling, also referred to as the reperfusion injury salvage kinase (RISK) pathway. With reference to Examples 4-6, in the HL-1 cardiomyocyte study, administration of abaloparatide and PTHrP(1-36) were both associated with better preservation of phospho-Akt and augmented expression of phospho-ERK following simulated ischemia-reperfusion. In the same study, co-administration of PD98059 (a pharmacologic inhibitor of MEK-ERK) attenuated the protective effect of abaloparatide and PTHrP(1-36), consistent with the concept that the better maintenance of HL-1 cell viability may be due in part to increased phosphorylation/activation of ERK. These cardioprotective results are particularly surprising in view of the previously noted concerns regarding adverse cardiovascular events observed in clinical trials and associated with administration of teriparatide and abaloparatide.

Accordingly, the present disclosure provides a method of treating cardiomyocyte reperfusion injury associated with an ischemic myocardial event. The method generally comprises administering to a subject in need thereof an effective amount of a PTH1R agonist, such as PTHrP(1-36), teriparatide, or abaloparatide. In particular embodiments, the PTH1R agonist is abaloparatide. Aspects of the method are described further herein below.

### Treatment of Cardiomyocyte Reperfusion Injury

The method as disclosed herein treats cardiomyocyte reperfusion injury.

Specifically, the method treats reperfusion injury associated with myocardial ischemia (i.e., an ischemic myocardial event). Any type of disease, disorder, injury, or procedure in which blood flow is temporarily reduced or stopped to a portion of the heart, then subsequently restored, is contemplated herein. Examples of types of reperfusion which may result in such reperfusion injury, and are therefore treated according to the disclosed method, include but are not limited to surgical procedures such as angioplasty, coronary artery bypass surgery, and cardiac transplant surgery, and non-surgical or surgically-related procedures, such as reversal of cardiopulmonary bypass, thrombolytic therapy, stent placement, and percutaneous coronary intervention.

In some embodiments, the method reduces an infarct volume relative to an infarct volume in a subject which has not been treated with an effective amount of a PTH1R agonist (e.g., abaloparatide).

In some embodiments, an expression level of phosphorylated Akt, phosphorylated ERK, or both, is enhanced relative to a corresponding expression level in the absence of the administration of a PTH1R agonist. (e.g., abaloparatide).

### Ischemic Myocardial Event

The method as disclosed herein treats cardiomyocyte reperfusion injury associated with an ischemic myocardial event. Examples of ischemic myocardial events include, but are not limited to, acute myocardial infarction, atherosclerosis, coronary artery spasm, cardiopulmonary bypass, thromboembolism, and cardiac transplant surgery. In some embodiments, the ischemic myocardial event is an acute event. In some embodiments, the acute ischemic myocardial event is myocardial infarction, coronary artery spasm, cardiopulmonary bypass, thromboembolism, or cardiac transplant surgery. In some embodiments, the acute ischemic myocardial event is shock, such as that associated with trauma. In some embodiments, the ischemic myocardial event is chronic, such as atherosclerosis. In some embodiments, the ischemic myocardial event comprises a partial occlusion of one or more coronary arteries. In some embodiments, the ischemic myocardial event comprises a complete occlusion of one or more coronary arteries. In some embodiments, the ischemic myocardial event comprises stopping the heart for a period of time, either intentionally as part of a surgical procedure, or inadvertently (e.g., through heart attack, shock, etc.).

In some embodiments, the method comprises administering to a subject identified as at risk for myocardial infarction an effective amount of a PTH1R agonist (e.g., abaloparatide).

### Treatment of Toxic Insult to Cardiomyocytes

In other embodiments, the method as disclosed herein may be useful in avoiding cardiomyocyte injury associated with exposure to cardiotoxic agents such as doxorubicin. Specifically, in some embodiments, the method treats cardiotoxicity associated with administration of the chemotherapeutic agent doxorubicin. Doxorubicin is widely prescribed for the treatment of solid tumours (e.g., breast, ovary and gastrointestinal) and hematologic malignancies (e.g., lymphoma and leukemia) in both adults and children. While an effective antitumor agent, doxorubicin causes cumulative and dose-dependent cardiotoxicity, ranging from occult changes in myocardial structure and function to severe cardiomyopathy and congestive heart failure. Such effects may result in the need for cardiac transplantation or may be fatal. Accordingly, in some embodiments, the method comprises administering to a subject an effective dose of abaloparatide prior to exposure of the subject to doxorubicin, during exposure to doxorubicin, or both.

Treatment of Neuronal Hypoxia and/or Reperfusion Injury

In still other embodiments, the method as disclosed herein may be useful in avoiding neuronal injury associated with ischemia, reperfusion injury, of both, such as neuronal injury associated with a stroke, or a circulatory impairment affecting blood flow to one or more portions of the brain or nervous system. Accordingly, in some embodiments, the method comprises administering to a subject an effective dose of abaloparatide prior to the patient experiencing an ischemic event and/or reperfusion. In some embodiments, the method comprises administering to a subject an effective dose of abaloparatide in a delayed manner, e.g., after the subject has experienced an ischemic event and/or reperfusion. In some embodiments, the method comprises administering abaloparatide both before and after the subject experiences an ischemic event, reperfusion, or both. In some embodiments, the ischemia is associated with a stroke, an occlusion of an artery, or a cardiac event.

### Therapeutically Effective Amount

The term “therapeutically effective amount” as used herein refers to an amount of PTH1R agonist (e.g., abaloparatide) that is sufficient to elicit the required or desired therapeutic and/or prophylactic response, as the particular treatment context may require. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount will vary based on a variety of factors, including but not limited to the physiological condition of the subject (e.g., age, body weight, sex, disease type and stage, medical history, general physical condition, responsiveness to a given dosage, and other present medications), the route of administration, and whether the administration is acute or chronic.

Examples of therapeutically effective amounts of PTH1R agonist (e.g., abaloparatide) include, without limitation, about 10 μg to about 400 such as about 20 μg to about 300 μg, about 50 μg to about 250 μg, about 70 μg to about 200 μg, about 70 μg to about 100 μg, about 70 μg to about 90 μg, or about 75 μg to about 85 μg. In some embodiments, the therapeutically effective amount of PTH1R agonist (e.g., abaloparatide) is about 20 μg, about 40 μg, about 60 μg, about 80 μg, about 100 μg, about 120 μg, about 150 μg, about 200 μg, about 250 μg, or about 300 μg.

In some embodiments, the therapeutically effective amount comprises a dose sufficient to provide a maximum exposure of about 240 pmol/kg·min of PTH1R agonist (e.g., abaloparatide).

### Route of Administration

The route of administration of the PTH1R agonist (e.g., abaloparatide) may vary. Suitable routes include subcutaneous, intravenous, intraarterial, intracoronary, intraperitoneal, intramuscular, transdermal, intradermal, intramuscular, and combinations thereof.

In some embodiments, the PTH1R agonist (e.g., abaloparatide) is administered subcutaneously. In some embodiments, abaloparatide is administered subcutaneously in an amount from about 20 μg to about 400 μg. In some embodiments, abaloparatide is administered subcutaneously in an amount of about 80 μg. In some embodiments abaloparatide is delivered via a subcutaneous injection that delivers 80 μg abaloparatide, e.g., the device and formulation for the currently approved TYMLOS® abaloparatide injection product. In some embodiments, the device and formulation is disclosed in U.S. Pat. No. 7,803,770, issued Sep. 28, 2010; U.S. Pat. No. 8,148,333, issued Apr. 3, 2012; and U.S. Pat. No. 8,748,382, issued Jun. 10, 2014, all of which are expressly incorporated by this reference in their entirety.

In some embodiments, the PTH1R agonist (e.g., abaloparatide) is administered transdermally. In some embodiments, abaloparatide is delivered with a transdermal device. In some embodiments, the transdermal device is a transdermal microneedle patch that delivers from about 100 jag to about 300 μg abaloparatide. In some embodiments, abaloparatide is delivered transdermally with a transdermal device, applicator, and/or formulation disclosed in International Patent Application Publication Nos. WO2017/062922, published 13 Apr. 2017; WO2017/184355, published 26 Oct. 2017; WO2017/062727, published 13 Apr. 2017; WO2017/184355, published 26 Oct. 2017; WO2019/077519, published Apr. 24, 2019; and WO2020/17443, published Sep. 30, 2020, all of which are expressly incorporated by this reference in their entirety.

In some embodiments, the PTH1R agonist (e.g., abaloparatide) is administered as a depot formulation, which may be subcutaneous, intradermal, or intramuscular.

In some embodiments, the PTH1R agonist (e.g., abaloparatide) is administered intravenously. In some embodiments, abaloparatide is administered intravenously at a dose sufficient to provide a maximum exposure of about 240 pmol/kg·min.

### Timing of Administration

The PTH1R agonist (e.g., abaloparatide) may be administered before, during, or after reperfusion, or any combination thereof. The PTH1R agonist (e.g., abaloparatide) may be administered acutely, chronically, or both. For example, abaloparatide may be administered prior to performing reperfusion, concurrent with performing reperfusion, or both prior to performing reperfusion and concurrently with performing reperfusion. In some embodiments, the abaloparatide is administered for a period of time after the reperfusion. In each case, PTH1R agonist (e.g., abaloparatide) may be administered as a single dose (acute) or multiple doses (chronic) by any of the routes disclosed herein above, and at any of the doses disclosed herein above.

In some embodiments, abaloparatide is administered to a patient in need thereof for at least a portion of the duration of a hospital stay, for example, from admittance to discharge, from admittance to the performance of a reperfusion procedure, and may be continued following discharge.

Examples of a suitable timeframe for administration prior to performing reperfusion include, without limitation, from about a minute to about 1 week prior to performing the reperfusion. In some embodiments, abaloparatide is administered for longer than 1 week prior to performing the reperfusion, for example, on the order of multiple weeks or months. Such long-term pre-administration may be useful in, for example, non-acute conditions such as myocardial infarction without ST elevation, for planned elective surgical procedures, and the like. In some embodiments, abaloparatide is administered from about 1 minute to about 1 hour, or from about 1 hour to about 12 hours, or from about 12 hours to about 24 hours, or from about 24 hours to about 7 days prior to performing the reperfusion. In some embodiments, abaloparatide is administered for up to 90 days prior to performing reperfusion. In some embodiments, the abaloparatide is administered daily for about 1 day to about 7 days prior to performing reperfusion. In some embodiments, the abaloparatide is administered once per day. In some embodiments, the abaloparatide is administered more than once per day, such as twice, three times, or four times per day. In some embodiments, the abaloparatide is administered continuously, e.g. via a patch or other transdermal application, or continuous intravenous infusion.

In some embodiments, abaloparatide is administered concurrently with performing reperfusion. In some embodiments, prior to performing the reperfusion and concurrently with performing reperfusion.

In some embodiments, abaloparatide is administered following perfusion, either instead of or in addition to prior and/or concurrent with reperfusion. The period of time for which the abaloparatide is administered post-reperfusion may vary. For example, in some embodiments, abaloparatide is administered for a period from 1 day to 7 days, 14 days, 30 days, 60 days, 90 days, 180 days, 365 days, or more following reperfusion. Such dosing may be daily, every other day, weekly, monthly, or may be continuously provided, as for example, with a transdermal patch.

### Cardiac Transplant

In another aspect is provided a method of treating cardiomyocyte reperfusion injury associated with cardiac transplant. The method may comprise pre-treatment of a donor heart, recipient, or both. Optionally or in addition, the method may comprise treatment of a donor heart, recipient, or both at the time of reperfusion (e.g., after transplant). In some embodiments, the method comprises contacting a donor heart with an effective amount of abaloparatide prior to performing reperfusion on said donor heart, concurrent with performing reperfusion on said donor heart, or prior to performing reperfusion on said donor heart and concurrently with performing reperfusion on said donor heart. The timing, routes of administration, doses, and the like may be as disclosed herein above, or may be determined specifically according to the application. For example, higher concentrations of abaloparatide may be desirable when confined locally rather than administered systemically, such as may be achieved by perfusing the heart with a composition comprising abaloparatide.

### Combination Therapy

In any of the disclosed embodiments, it is contemplated that the administration of abaloparatide may be performed in combination with other treatment modalities, including any standard of care therapies of a pharmacological, surgical, or mechanical nature. For example, abaloparatide may be administered concurrently or sequentially with pharmacological agents including, but not limited to, antiarrhythmics, anticoagulants, β-adrenoceptor blockers, nitrates, angiotensin converting enzyme (ACE) inhibitors, thrombolytics, calcium channel blockers, diuretics, lidocaine, procainamide, amiodarone, atropine, epinephrine, and the like.

It will be readily apparent to one of ordinary skill in the relevant arts that suitable modifications and adaptations to the methods and applications described herein can be made without departing from the scope of any embodiments or aspects thereof. The methods provided are exemplary and are not intended to limit the scope of the claimed embodiments. All of the various embodiments, aspects, and options disclosed herein can be combined in all variations. The scope of the methods described herein include all actual or potential combinations of embodiments, aspects, options, examples, and preferences herein.

Embodiments of the present disclosure can be further defined by reference to the following non-limiting examples. It will be apparent to those skilled in the art that many modifications, both to materials and methods, can be practiced without departing from the scope of the present disclosure.

### EXEMPLIFICATION

### Example 1. Effect of Abaloparatide on Cardiomyocyte Viability

HL-1 cardiomyocytes were grown to ˜80% confluence in 95% room air/5% CO₂ at 37° C. in Claycomb medium supplemented with fetal bovine serum (10%), L-glutamine (2 mM), norepinephrine (0.1 mM), penicillin (100 U/mL) and streptomycin (100 μg/mL). The cardiomyocytes were then incubated for 24 hours with abaloparatide (0.1 nM, 1.0 nM, 10 nM, 100 nM) or vehicle control. At 24 hours after the onset of incubation, the cells were subjected to 2 hours of simulated ischemia (achieved by depletion of oxygen and nutrients), followed by 24 hours of reoxygenation. During simulated ischemia, Claycomb medium containing drug/placebo was replaced with drug-free hypoxia buffer at pH 6.6 (composed of NaCl (125 nM), KCl (8 mM), KH₂PO₄ (1.2 mM), 1.25 mM MgSO₄ (1.25 mM), CaCl₂ (1.2 mM), NaHCO₃ (6.25 mM), HEPES (20 mM), glucose (5.5 mM), 2-deoxy-D-glucose (20 mM), sodium lactate (5 mM): all ingredients from Sigma Aldrich, St. Louis, MO, USA) and the culture plates were sealed in a hermetic chamber with GasPak EZ Gas Generating Sachets (BD Biosciences, San Jose, CA, USA). Reoxygenation was achieved by removing the plates from the sealed chamber and exchanging the hypoxia buffer with serum-free Claycomb medium. Additional time- and treatment-matched normoxic cultures served as controls (n=4 independent replicates per group). Cell viability was quantified using the 3-(4,5-dimethythiazol-2-yl)-2,5-diphenyl tetrazolium bromide (MTT) assay.

### Results

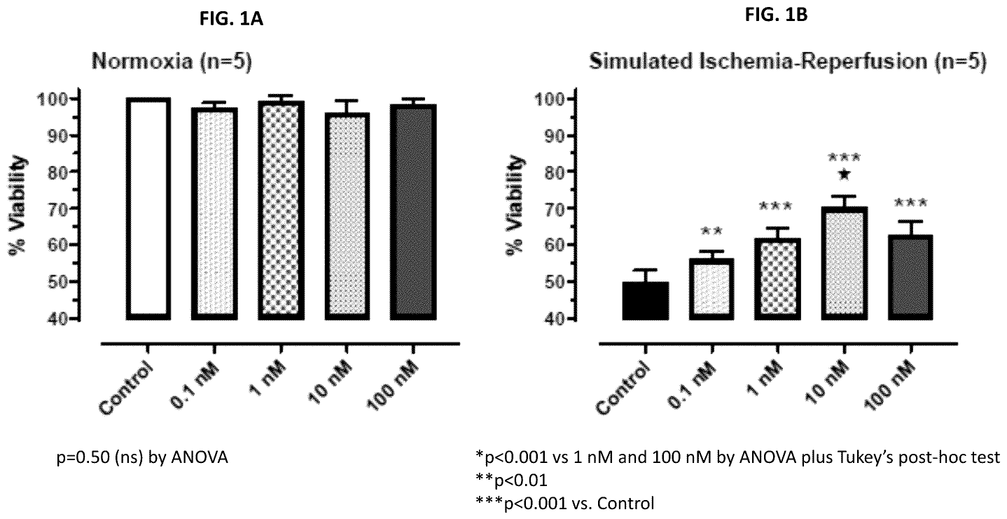

Under normoxic conditions, abaloparatide had no effect on HL-1 cell viability as illustrated in FIG. 1A. For cells subjected to the simulated ischemia-reperfusion, viability was better-maintained with abaloparatide treatment as compared with vehicle control, with the greatest efficacy seen at a concentration of 10 nM of abaloparatide (FIG. 1B).

### Example 2. Effect of PTHrP(1-36) on Cardiomyocyte Viability

HL-1 cardiomyocytes were grown to ˜80% confluence as in Example 1, and incubated for 24 hours with 10 nM and 100 nM human parathyroid hormone-related protein (PTHrP(1-36)) or vehicle control before being subjected to simulated ischemia. At 24 hours after the onset of incubation, the cells were subjected to 2 hours of simulated ischemia (achieved by depletion of oxygen and nutrients), followed by 24 hours of reoxygenation. Additional time- and treatment-matched normoxic cultures served as controls. Cell viability was quantified using the 3-(4,5-dimethythiazol-2-yl)-2,5-diphenyl tetrazolium bromide (MTT) assay.

### Results

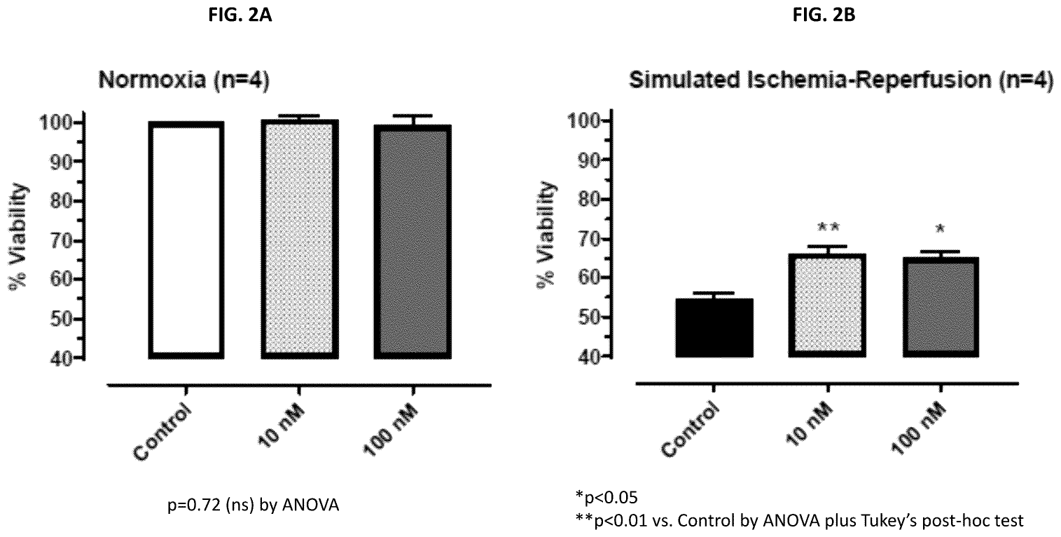

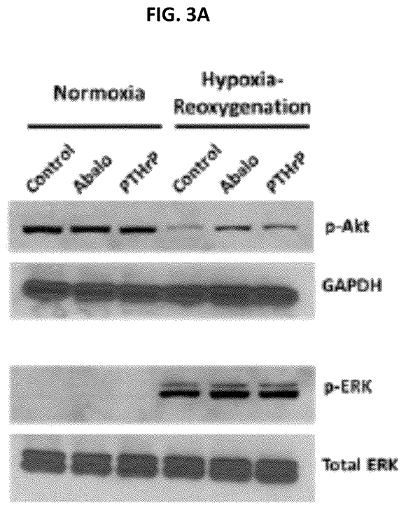

Under normoxic conditions, PTHrP had no effect on HL-1 cell viability as illustrated in FIG. 2A. For cells subjected to simulated ischemia-reperfusion, viability was better-maintained with PTHrP treatment when compared with vehicle-controls, with no statistical difference in efficacy observed between the 10 and 100 nM concentrations (FIG. 2B).

### Example 3. Effect of Abaloparatide and PTHrP(1-36) on Expression of Survival Kinases

The object of this study was to obtain insight into the effect of PTHrP(1-36) and abaloparatide on the expression of phospho-ERK and phospho-Akt in HL-1 cardiomyocytes subjected to simulated ischemia-reperfusion. In Example 3A, HL-1 cells, grown to −80% confluence, were incubated for 24 h with PTHrP(1-36) (10 nM), abaloparatide (10 nM) or a vehicle as described In Examples 1 and 2. At 24 h after the onset of treatment, cells underwent 2 h of SI+10 min reoxygenation, while additional time- and treatment-matched normoxic cultures served as controls (n=3 independent replicates per group). At 10 min post-reoxygenation, cells were harvested and lysed in RIPA buffer containing 2 protease and phosphatase inhibitors. The lysates were centrifuged at 14,000×g for 15 min, and the resultant supernatant was collected and probed for the expression of ERK (total and phosphorylated: antibodies from Cell Signaling Technology Inc., Danvers, MA, USA), PI3 kinase/Akt (total and phosphorylated: Cell Signaling Technology Inc., Danvers, MA, USA) and GAPDH (Sigma Aldrich Inc., St. Louis, MO, USA) using standard methods Immunoreactive bands were identified by incubation with horseradish peroxidase (HRP)-conjugated secondary antibody, visualized using x-ray film, and quantified using the NIH ImageJ software. In Example 3B, HL-1 cardiomyocytes were incubated for 24 h with PTHrP(1-36) (10 nM), abaloparatide (10 nM) or a vehicle, with or without the addition of PD98059 (pharmacologic inhibitor of MEK-ERK: 5 μM, Cell Signaling Technology Inc., Danvers, MA, USA). At 24 h after the onset of treatment, cells underwent either 2 h of SI+24 h reoxygenation or a time-matched period of normoxia (n=2 independent replicates per group). At 24 h following relief of SI, cell viability was quantified using the MTT assay as described in Example 1.

### Results

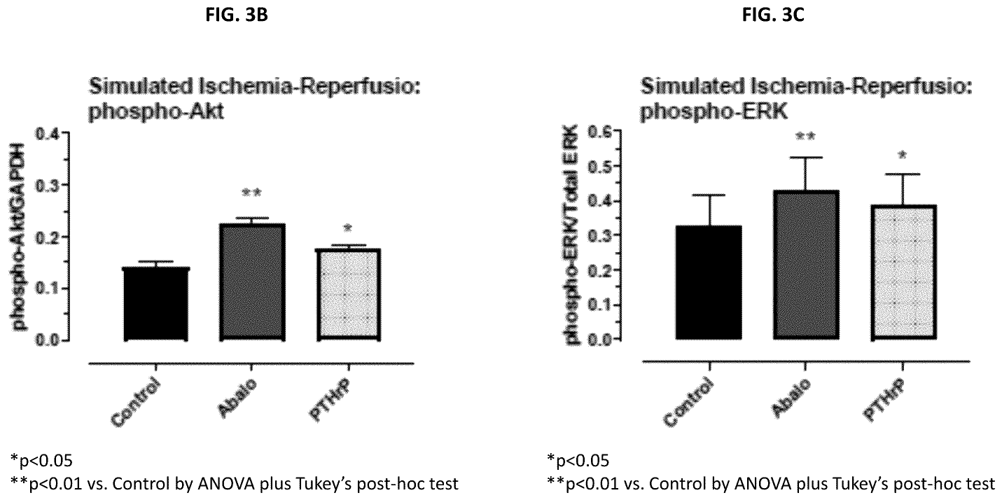

Under normoxic conditions, all Akt was present in its phosphorylated form, while all ERK was unphosphorylated, with no differences among groups (FIG. 3A). For quantitation, expression of phospho-Akt was normalized to GAPDH, and expression of phospho-ERK was normalized to total ERK. In vehicle-treated cells, simulated ischemia-reperfusion was accompanied by a decrease in expression of phospho-Akt (to <50% of values seen in normoxic controls) and the presence of phospho-ERK, while abaloparatide and PTHrP treatment were associated with better preservation of phospho-Akt and augmented expression phospho-ERK (FIGS. 3B and 3C, respectively).

### Example 4. Concurrent Assessment of Abaloparatide and PTHrP(1-36) on Cardiomyocyte Viability

HL-1 cardiomyocytes were grown to ˜80% confluence and incubated for 24 hours with 10 nM and 100 nM PTHrP(1-36), 10 nM and 100 nM Abaloparatide, or vehicle control. At 24 hours after the onset of incubation, the cells were subjected to 2 hours of simulated ischemia (achieved by depletion of oxygen and nutrients), followed by 24 hours of reoxygenation. Additional time- and treatment-matched normoxic cultures served as controls. Cell viability was quantified using the 3-(4,5-dimethythiazol-2-yl)-2,5-diphenyl tetrazolium bromide (MTT) assay.

### Results

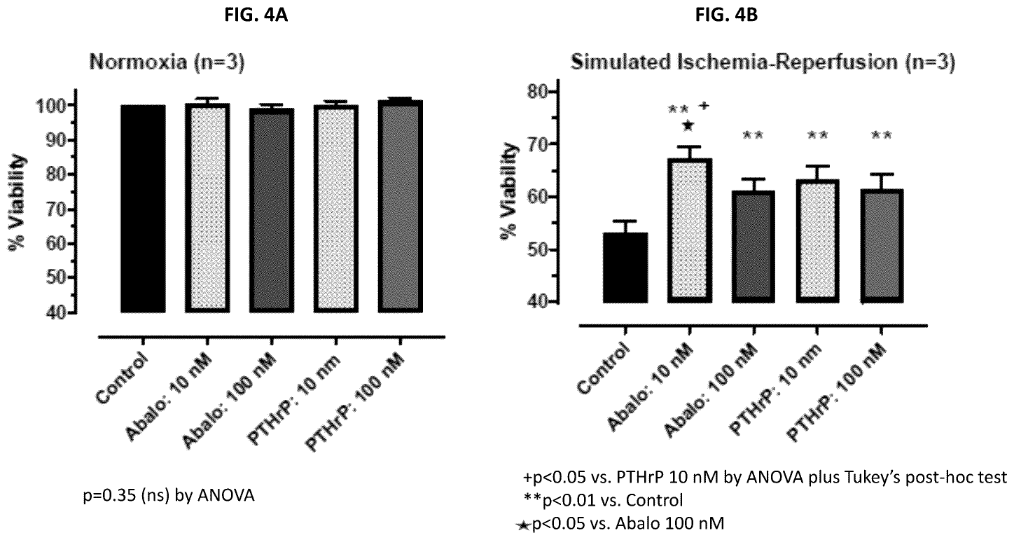

Under normoxic conditions, neither agent had an effect on HL-1 cell viability as illustrated in FIG. 4A. For cells subjected to simulated ischemia-reperfusion, viability was better-maintained with both abaloparatide and PTHrP treatment when compared with vehicle-controls as illustrated in FIG. 4B. The viability with 10 nM abaloparatide was greater than that achieved with 100 nM abaloparatide and 10 nM PTHrP(1-36) (p<0.05 between groups; FIG. 4B).

### Example 5. Effect of Co-Administration of PD98059 on the Cardioprotective Effect of Abaloparatide and PTHrP(1-36)

HL-1 cardiomyocytes were incubated for 24 hours with 10 nM abaloparatide, 10 nM PTHrP(1-36), or vehicle control, with or without the addition of 5 μM PD98059 (a pharmacologic inhibitor of MEK-ERK). Twenty-four hours after the onset of treatment, cells underwent 2 hours of simulated ischemia followed by 24 hours of reoxygenation. Additional time- and treatment-matched normoxic cultures served as controls. Cell viability was quantified using the MTT assay.

### Results

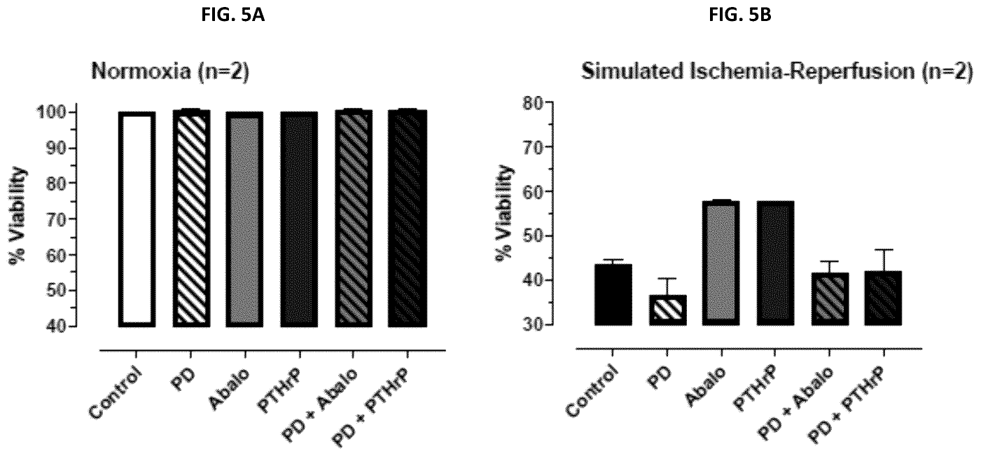

Under normoxic conditions, none of the agents, alone or in combination, had an effect on HL-1 cell viability (FIG. 5A). Consistent with results in the previous examples, viability was better maintained with both abaloparatide and PTHrP treatment when compared with vehicle-controls for cells subjected to simulated ischemia-reperfusion, and the protective effects of abaloparatide and PTHrP were attenuated by co-administration of PD98059 (FIG. 5B).

### Example 6. Effect of Co-Administration of PD98059 on ERK Expression in Abaloparatide and PTHrP(1-36) Treated Cells

HL-1 cardiomyocytes were treated as described in Example 5, but were harvested at 10 minutes post-reoxygenation, lysed, and probed for phospho- and total ERK expression as detailed in Example 5.

### Results

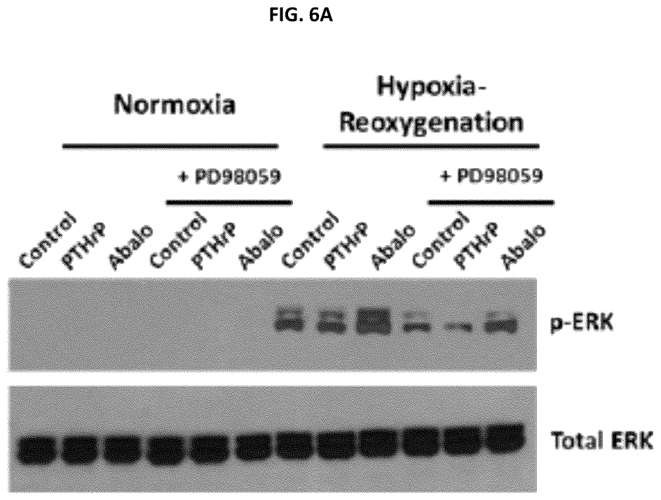

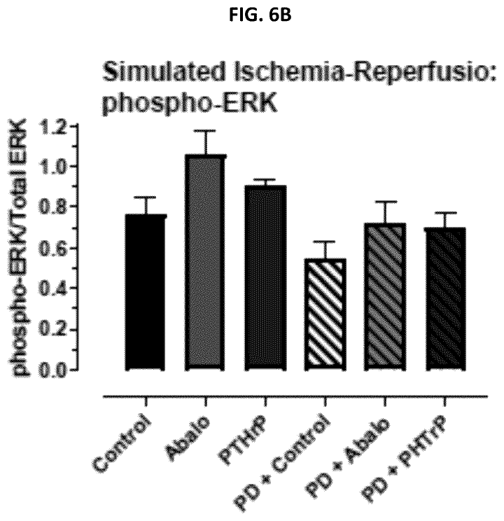

Consistent with Example 5 results, under normoxic conditions, no phospho-ERK was detected (FIG. 6A). In cells subjected to simulated ischemia-reperfusion, abaloparatide and PTHrP treatment were associated with augmented expression phospho-ERK when compared with vehicle-controls, and phospho-ERK expression in all groups (control, abaloparatide- and PTHrP-treated) was attenuated by co-administration of PD98059 (FIG. 6B).

### Example 7. Pharmacokinetics of PTHrP(1-36) Following Administration of Peptide

A study was performed to determine the temporal profile of PTHrP(1-36) in circulating blood following administration of PTHrP(1-36). Female domestic swine (n=3; body weight=˜30 kg, purchased from the Michigan State University Swine Farm) were pretreated with carprofen (4.4 mg/kg PO) and anesthetized with an intramuscular injection of midazolam (0.4 mg/kg)+xylazine (1.0 mg/kg)+butorphanol (0.2 mg/kg). The pigs were then intubated and ventilated with room air, with anesthesia maintained by the inhalation of isoflurane (1.5-2%). Under sterile conditions, a surgical cut-down was performed and a cannula was inserted in the right jugular vein. After obtaining an initial baseline blood sample (volume of 3 mL), 15 μg PTHrP(1-36) was dissolved in 500 μL sterile saline and administered by subcutaneous injection. Subsequent serial blood samples were obtained at 5, 10, 15, 30, 45, 60, 90 and 120 min post-treatment. All samples were collected in vacutainers containing EDTA as the anticoagulant, and aprotinin (100 μL per mL of blood) was added to each sample immediately after collection. Samples were centrifuged for 12 min at 1500×g and the plasma was collected and stored at −80° C. until analyzed. After obtaining the final blood sample, the jugular cannula was removed, the incision was sutured, and anesthesia was discontinued. Animals were returned to their cages, allowed to recover for 1-2 weeks, and subsequently enrolled in the experimental protocol of Example 8. The primary endpoint, plasma PTHrP concentration (in ng/mL) was quantified using a standard commercial ELISA kit as per the manufacturer's instructions (Phoenix Pharmaceuticals Inc., Burlingame, CA, USA, Catalog #EK-056-04), with all samples assayed in duplicate.

### Results

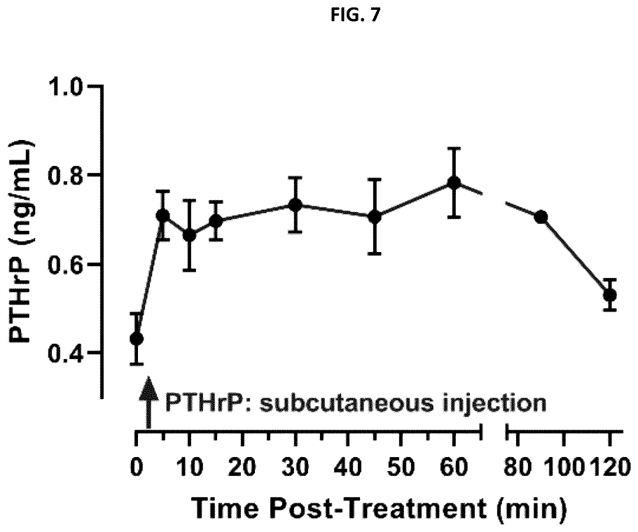

Endogenous plasma PTHrP concentrations measured at baseline, before administration of exogenous peptide, averaged 0.43 ng/mL (FIG. 7). Plasma PTHrP concentration increased rapidly following injection (mean of 0.71 ng/mL at 5 min post-injection) and remained approximately constant throughout the initial 90 min post-treatment (FIG. 7). This temporal profile suggests that, for experiments conducted in Example 8 below, PTHrP(1-36) concentrations were increased at the onset of coronary occlusion and remained elevated throughout ischemia and the initial minutes of reperfusion.

### Example 8. PTHrP Infarct Size Study

A study was conducted in female pigs to assess the effect of PTHrP in a coronary artery occlusion-reperfusion model. The primary study endpoint was myocardial infarct size (quantified by triphenyltetrazolium staining) and expressed as a % of the ‘area at risk’: i.e., volume of myocardium rendered ischemic during coronary occlusion (delineated by injection of Unisperse Blue pigment into the coronary circulation). Analysis was conducted in a blinded manner, without knowledge of the group assignments.

Fifteen female pigs (from Example 7) were assigned to receive PTHrP (1 μg/kg PTHrP(1-36) in sterile saline) or vehicle (sterile saline alone). PTHrP or placebo were administered once per day for 3 days via subcutaneous injection, with a 4th dose administered approximately 5 minutes before the onset of coronary artery occlusion.

All animals were anesthetized as described in Example 7. Core temperature was maintained at 38° C. using a Blanketrol® heating pad (Gentherm, Cincinnati, OH, USA), and hemodynamics, spO₂ and end-expiratory CO₂ were monitored throughout each experiment (SurgiVet®: Smiths Medical Inc., Minneapolis, MN, USA). The left jugular vein was cannulated for administration of fluids, the heart was exposed via a midline sternotomy, and a segment of the left anterior descending coronary artery (LAD) was isolated, typically midway along its course and distal to the first major diagonal branch.

After stabilization, pigs received the final dose of PTHrP(1-36) or a vehicle and, 5 min later, the LAD was occluded by placing an atraumatic vascular clamp on the isolated arterial segment. At 10-12 min post-occlusion, lidocaine (1.3 mg/kg) was administered via the jugular vein in an effort to minimize the incidence of lethal ventricular fibrillation (VF). At 75 min post-occlusion, pigs received an additional dose of lidocaine, and the LAD was reperfused by removal of the vascular clamp. For animals that developed VF at any time during the protocol, resuscitation was attempted by applying DC countershocks directly to the heart (energy of 20-50 Joules; maximum of 4 attempts). At 3 h post-reperfusion, the LAD was ligated at the site of the previous occlusion and Unisperse blue pigment (Ciba Specialty Chemicals Corp., Tarrytown, NY, USA) was injected IV to delineate the area at risk (AR), or volume of myocardium rendered ischemic during coronary occlusion. Pigs were then euthanized under deep anesthesia by IV injection of Fatal-Plus® (Vortech Pharmaceuticals Ltd., Southfield, MI, USA). The hearts were rapidly excised, cut into 5-6 transverse slices and photographed. The heart slices were immediately incubated in triphenyltetrazolium chloride (Sigma Aldrich, St. Louis, MO, USA; 10 min at 37° C.) to discern infarcted versus viable tissue and re-photographed.

The primary endpoint was the quantitative assessment of myocardial infarct size. The area at risk (AR) and area of necrosis (AN) in each heart slice were measured from the photographs using ImageJ, corrected for tissue weight, and summed for each heart. AR was expressed as a % of the total left ventricular (LV) weight, and AN was expressed as a % of the AR [27]. In addition, heart rate and arterial pressure were and tabulated at baseline and at 15 min, 1 h and 3 h post-reperfusion, and the incidence of VF was recorded for each pig.

### Results

Twelve of the fifteen pigs developed one or more episodes of ventricular fibrillation (VF) between 13-73 minutes into left anterior descending (LAD) coronary artery occlusion or immediately upon reperfusion (n=7 in the PTHrP-treated group and n=5 in the Vehicle-Control cohort). Nine of the twelve pigs that developed VF were successfully resuscitated by application of one to three 25 Joule DC shocks directly to the heart (n=5 that received PTHrP and n=4 Vehicle-Controls). Three pigs that developed VF (1 Control, 2 PTHrP-treated) failed to respond to defibrillation and died during coronary artery occlusion. The remaining 12 pigs (n=6 treated with PTHrP, n=6 controls) successfully underwent 75 minutes of LAD occlusion and 3 hours of reperfusion and completed the protocol.

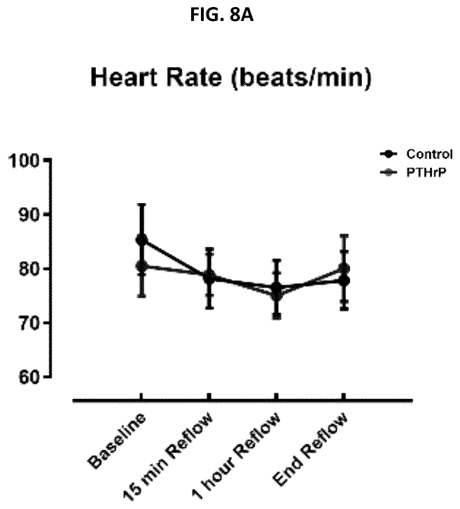

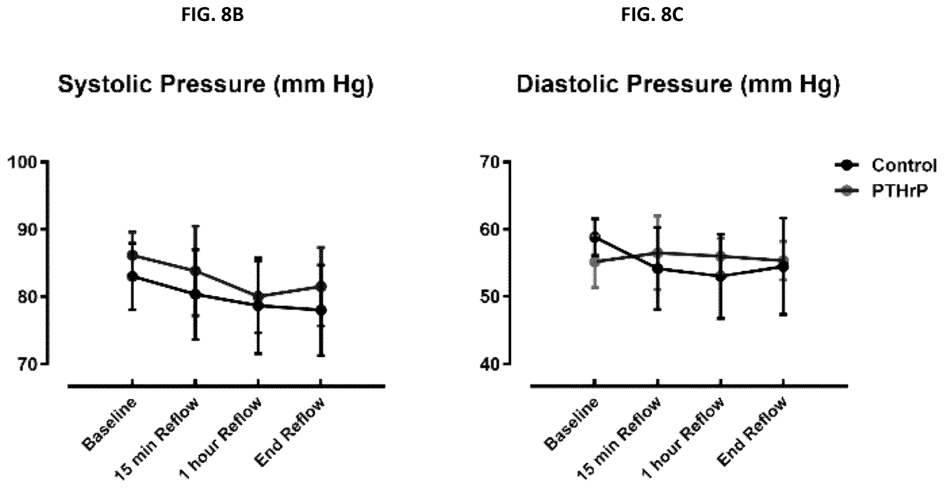

Heart rate, systolic, and diastolic pressures were measured at 15 min, 1 h and 3 h (end) following reperfusion in PTHrP(1-36)-treated pigs and vehicle-controls. Data are provided in FIGS. 8A-8C, respectively), reported as mean±SEM. With reference to FIGS. 8A-8C, heart rate, systolic and diastolic pressure were comparable in control and PTHrP(1-36)-treated pigs at baseline and throughout reperfusion No differences in heart rate or arterial pressures between PTHrP(1-36) and placebo-treated pigs was observed. This is in apparent contrast to the hemodynamic effects of abaloparatide administered to post-menopausal women with osteoporosis, where subcutaneous injection of the agent was associated with a modest and transient increases in heart rate and decreases in supine blood pressure (Cosman et al., “Cardiovascular Safety of Abaloparatide in Postmenopausal Women With Osteoporosis: Analysis From the ACTIVE Phase 3 Trial” J. Clin. Endocrinol Metab. 2020, 105, 3384-3395). Without wishing to be bound by theory, it is believed that this discrepancy may be due to species differences and/or the isoflurane anesthesia utilized in the present study. Most importantly, there was no evidence of an increase in the incidence of adverse cardiac events in subjects treated with the PTHrP analog. In addition, area at risk, expressed as a % of total left ventricular weight, did not differ between groups, averaging 16.9±1.1% and 17.6±1.0% in the control and PTHrP(1-36)-treated groups, respectively (p=0.60 (ns); not shown).

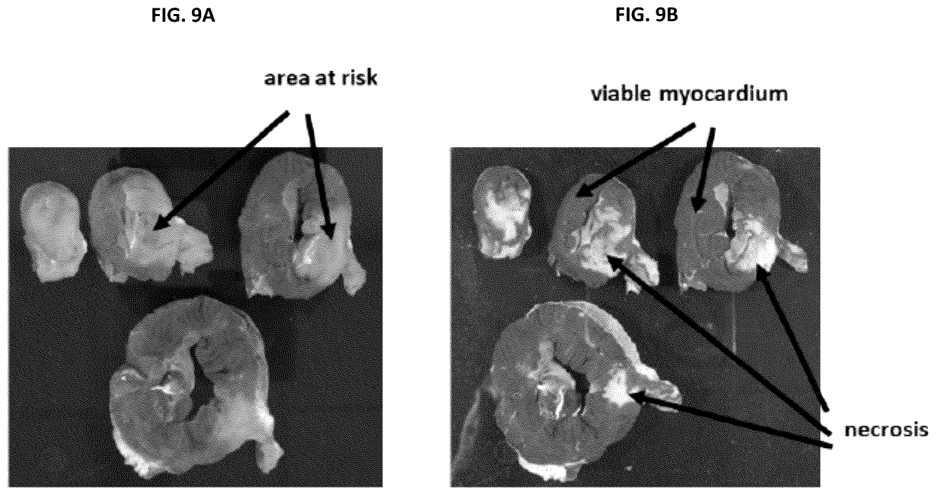

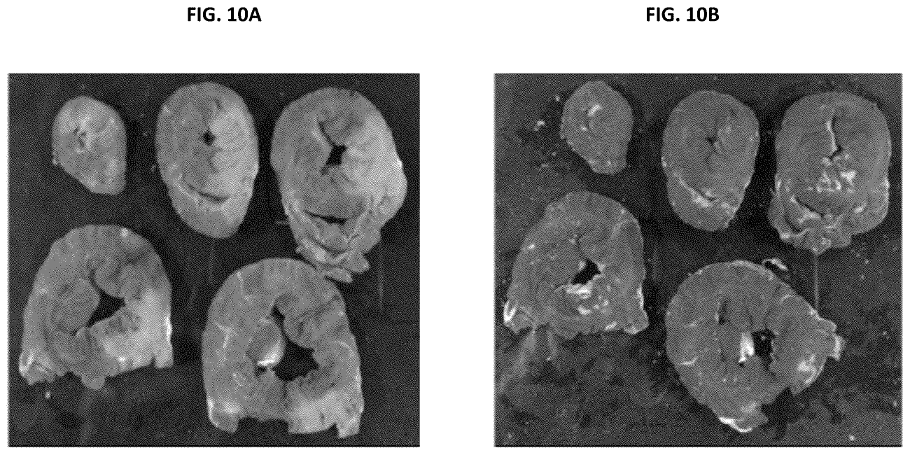

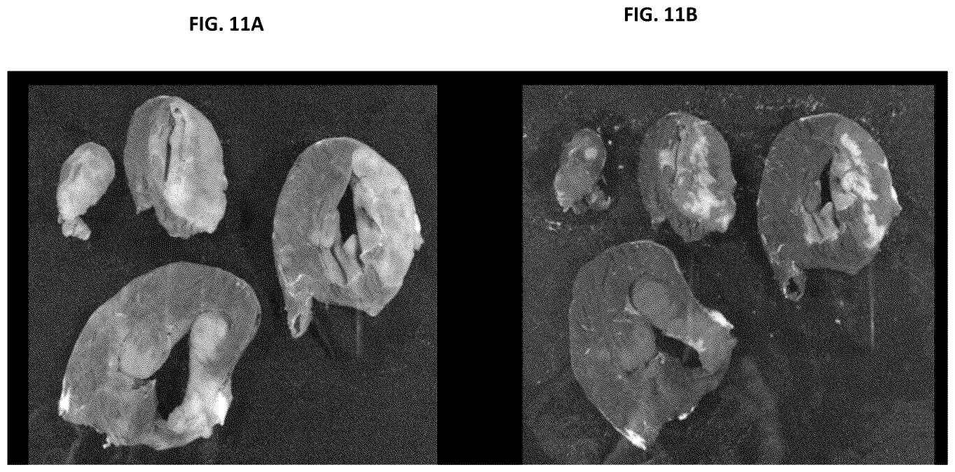

Transverse left ventricular slices from one control and two PTHrP-treated pigs following completion of the protocol were obtained, and the area at risk was delineated by injection of Unisperse blue pigment (FIGS. 9A, 10A, and 11A, respectively). The volume of myocardium rendered ischemic during occlusion is devoid of dye and appears pink. The area of necrosis was delineated by triphenyltetrazolium staining. Viable myocardium stained red, while infarcted myocardium remained unstained and appeared pale (FIGS. 9B, 10B, and 11B for the control and two PTHrP-treated pigs, respectively).

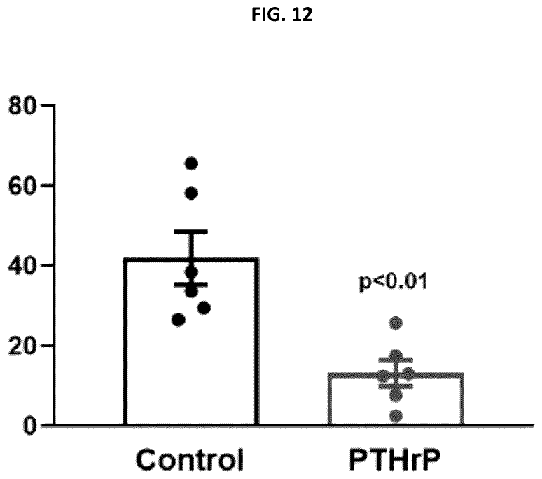

Data for the full protocol set is provided in FIG. 12, which shows that the infarct sizes ranged from 27% to 66% of the risk region in the control group and from 3% to 26% of the area at risk in pigs that received PTHrP. Mean infarct size in the control cohort was 42.0±6.6% of the area at risk (AR). In contrast, area of necrosis (AN)/AR was significantly reduced in pigs pretreated with PTHrP(1-36) at an average of 13.1±3.3% (p<0.01; FIG. 12). Accordingly, the mean infarct size was significantly smaller in the PTHrP-treated group versus the control group.

### Example 9. Efficacy of Delayed Treatment with Abaloparatide in HL-1 Cardiomyocytes

The goal of this study is to establish whether the efficacy of abaloparatide treatment is maintained (or, potentially, attenuated) when the agent is given 10 min before the onset of ischemia or, more importantly, immediately at the time of reoxygenation. HL-1 cells will be grown to 80% confluence using our standard protocols. Twelve groups of cells will undergo 2 hours of simulated ischemia (achieved by depletion of oxygen and nutrients)+24 hours reoxygenation, and will be assigned to receive the following, respectively:
1. Abalo (10 nM) dissolved in Claycomb medium (CM) and administered 24 hr before the onset of simulated ischemia (SI)
2. Abalo (100 nM) dissolved in CM and administered 24 hr before the onset of SI
3. Volume-matched CM vehicle administered 24 hr before the onset of SI
4. Abalo (10 nM) dissolved in CM and administered 10 min before the onset of SI
5. Abalo (100 nM) dissolved in CM and administered 10 min before the onset of SI
6. Volume-matched CM vehicle administered 10 min before the onset of SI
7. Abalo (10 nM) dissolved in CM and administered immediately at reoxygenation
8. Abalo (100 nM) dissolved in CM and administered immediately at reoxygenation
9. Volume-matched CM vehicle administered immediately at reoxygenation
10. Abalo (10 nM) dissolved in CM and administered 10 min post-reoxygenation
11. Abalo (100 nM) dissolved in CM and administered 10 min post-reoxygenation
12. Volume-matched CM vehicle administered 10 min post-reoxygenation

An additional three groups will serve as time-matched normoxic controls, receiving, respectively:
13. Abalo (10 nM) dissolved in CM and administered for 50 hours (corresponding to 24 hr pretreatment+2 hr simulated ischemia+24 hr reoxygenation)
14. Abalo (100 nM) dissolved in CM and administered for 50 hours
15. Volume-matched CM vehicle administered for 50 hours

Cell viability will be quantified using the standard MTT [3-(4,5-dimethythiazol-2-yl)-2,5-diphenyl tetrazolium bromide] assay. For each group, viability data will be obtained from 5 independent replicates. It is expected, based on prior results, that % cell death following simulated ischemia-reperfusion in vehicle-treated cells will average ˜50%, and that abaloparatide will have no effect on the viability of normoxic HL-1 cells in cells subjected to simulated ischemia+reoxygenation, and that viability will be significantly better-maintained in groups that are pretreated for 24 hours with abalo (HL-1 cell death reduced to −30-40%).

### Example 10. Effect of Abaloparatide on Doxorubicin-Induced Cardiotoxicity

The goal of this study is to explore the protective effect of abaloparatide on HL-1 cardiomyocyte exposed to doxorubicin. HL-1 cells will be grown to 80% confluence using our standard protocols. Five groups of cells will be incubated in doxorubicin (dox: 2.5 μM) for 24 hours—a concentration previously shown to kill ˜50% of HL-1 cardiomyocytes. Four groups will be pretreated with abalo (0.1 nM, 1.0 nM, 10 nM or 100 nM) initiated 24 hours before exposure to doxorubicin. An additional 2 groups will serve as time-matched controls: 1 will receive 100 nM abalo pretreatment followed by 24 hours of exposure to dox-vehicle, and the second will receive abalo-vehicle pretreatment followed by 24 hours of exposure to dox-vehicle. Cell viability will be quantified using the MTT [3-(4,5-dimethythiazol-2-yl)-2,5-diphenyl tetrazolium bromide] assay. For each group, data will be obtained from 5 independent replicates.

### Example 11. Efficacy of Pretreatment with Abaloparatide in HT22 Neurons

The goal of this study is to demonstrate that the protective effect of abaloparatide is not limited to cardiomyocytes, and to identify a dose of abaloparatide that attenuates neuronal cell death. HT22 cells will be grown to 70% confluence using standard protocols. To confirm expression of functional PTH1R in HT22 neurons, cells will be incubated for 24 hours with abaloparatide (10 nM) or vehicle. Intracellular cAMP will be assessed by ELISA. Four concentrations of abaloparatide will be evaluated: 0.1 nM, 1.0 nM, 10 nM and 100 nM. Cells will undergo 1 hour of simulated ischemia (i.e., achieved by depletion of oxygen and nutrients)+24 hours reoxygenation. Additional time-matched normoxic cultures will serve as controls. Cell viability will be quantified using the MTT [3-(4,5-dimethythiazol-2-yl)-2,5-diphenyl tetrazolium bromide] assay. Additional experiments may be conducted in which abaloparatide is administered to HT22 cells in a delayed manner to study the efficacy of delayed treatment with abaloparatide in HT22 neurons.

What is claimed is:

1. A method of treating cardiomyocyte reperfusion injury associated with an ischemic myocardial event, the method comprising administering to a subject in need thereof an effective amount of abaloparatide prior to performing reperfusion, concurrent with performing reperfusion, or prior to performing reperfusion and concurrently with performing reperfusion.

2. The method of claim 1, wherein the ischemic myocardial event is an acute myocardial infarction, atherosclerosis, coronary artery spasm, cardiopulmonary bypass, thromboembolism, or cardiac transplant surgery, and wherein the ischemic myocardial event comprises at least a partial occlusion of one or more coronary arteries.

3. The method of claim 1, wherein the reperfusion comprises angioplasty, stent placement, intra-aortal balloon procedure, coronary artery bypass surgery, or reversal of cardiopulmonary bypass.

4. The method of claim 1, wherein the administration is subcutaneous, intravenous, intradermal, intraarterial, transdermal, intraperitoneal, intramuscular, or intracoronary.

5. The method of claim 1, wherein the abaloparatide is administered from about 1 minute to about 1 hour, or from about 1 hour to about 12 hours, or from about 12 hours to about 24 hours, or from about 24 hours to about 7 days, or from about 7 days to about 30 days, or from about 30 days to about 90 days prior to performing the reperfusion.

6. The method of claim 1, wherein the abaloparatide is administered once daily for about 1 day to about 7 days prior to performing reperfusion.

7. The method of claim 1, wherein the abaloparatide is administered concurrently with performing reperfusion.

8. The method of claim 1, wherein the abaloparatide is dosed subcutaneously in an amount from about 20 μg to about 400 μg.

9. The method of claim 1, wherein the abaloparatide is dosed subcutaneously in an amount of about 80 μg.

10. The method of claim 1, wherein the abaloparatide is administered using a transdermal microneedle patch in an amount from about 100 μg to about 300 μg.

11. The method of claim 1, wherein the abaloparatide is administered intravenously at a dose sufficient to provide a maximum exposure of about 240 pmol/kg·min.

12. The method of claim 1, wherein the subject is identified as at risk for myocardial infarction.

13. The method of claim 1, wherein an expression level of phosphorylated Akt, phosphorylated ERK, or both, is enhanced relative to a corresponding expression level in the absence of the administration of abaloparatide.

14. A method of treating cardiomyocyte reperfusion injury associated with cardiac transplant, the method comprising contacting a donor heart with an effective amount of abaloparatide prior to performing reperfusion on said donor heart, concurrent with performing reperfusion on said donor heart, or prior to performing reperfusion on said donor heart and concurrently with performing reperfusion on said donor heart.

15. The method of claim 14, comprising perfusing the donor heart with a composition comprising abaloparatide prior to revitalization.


SEQUENCE LISTING (1 sequence)

SEQ ID NO: 1 - protein, 34 aa, synthetic construct
  MOD_RES 29: note = 2-aminoisobutyric acid
  MOD_RES 34: note = amidation
AVSEHQLLHDKGKSIQDLRRRFFLHHLIAEIHTA